(19) United States
(12) Patent Application Publication
(10) Pub. No.: US 2022/0254493 A1
(43) Pub. Date: Aug. 11, 2022

(54) CHRONIC DISEASE PREDICTION SYSTEM BASED ON MULTI-TASK LEARNING MODEL

(71) Applicant: ZHEJIANG UNIVERSITY, HANGZHOU, Zhejiang Province (CN)

(72) Inventors: JIAN WU, HANGZHOU, ZHEJIANG PROVINCE (CN); XIAOHONG JIANG, HANGZHOU, ZHEJIANG PROVINCE (CN); HAOCHAO YING, HANGZHOU, ZHEJIANG PROVINCE (CN); RUIWEI FENG, HANGZHOU, ZHEJIANG PROVINCE (CN); XUECHEN LIU, HANGZHOU, ZHEJIANG PROVINCE (CN); YAN CAO, HANGZHOU, ZHEJIANG PROVINCE (CN)

(21) Appl. No.: 17/623,555

(22) PCT Filed: Nov. 12, 2020
(86) PCT No.: PCT/CN2020/128427

§ 371 Date: Dec. 28, 2021

(30) Foreign Application Priority Data
Dec. 19, 2019 (CN) 201911317824.0

(51) Int. Cl. G16H 50/20

(52) U.S. Cl. CPC G16H 50/20

(57) ABSTRACT

A chronic disease prediction system based on a multi-task learning model. The system includes a computer memory, a computer processor and a computer program which is stored in the computer memory and executable on the computer processor, wherein a trained chronic disease prediction model is stored in the computer memory, and the chronic disease prediction model is composed of a shared layer convolutional neural network and a plurality of chronic disease branch networks; and when executing the computer program, the computer processor implements the following steps: preprocessing a to-be-predicted physical examination record and then inputting the record into the shared layer convolutional neural network of the chronic disease prediction model for feature extraction to obtain a feature map, and inputting the obtained feature map into each chronic disease branch network and performing feature extraction and prediction respectively to obtain a chronic disease prediction result.

### FIELD OF TECHNOLOGY

The present invention relates to the technical field of artificial intelligence in medicine, and in particular to a chronic disease prediction system based on a multi-task learning model.

### BACKGROUND TECHNOLOGY

Chronic diseases are a type of latent and long-term common diseases, including diabetes, cardiovascular diseases, cancers and respiratory diseases. In recent years, the number of patients with chronic diseases is increasing rapidly. Generally speaking, the causes of chronic diseases are complex, so continuous treatment is required. Therefore, chronic diseases bring harm to people's health and life, and the death rate and treatment burden are continuously increasing. If the chronic diseases can be discovered and intervened early, these problems can be effectively alleviated.

At present, there have been some methods which try to discover and treat chronic diseases as early as possible. These methods may be generally divided into two categories: one category is to focus on researching data containing people's living habit and demographic variable so as to find out body conditions or living habits which may cause a certain chronic disease, thereby preventing the chronic disease.

For example, Chinese patent document with the publication number CN107153774A discloses construction of a chronic disease risk assessment hyperbolic model and a disease prediction system applying the model. It relies on the longitudinal health management data of more than 20 health management centers in Shandong Province to build a Shandong multi-center health management longitudinal observation queue, discuss the effect of heredity, environment, personal lifestyle and health intervention factor in the occurrence, development and prognosis processes of major chronic diseases, establish a risk assessment hyperbolic model and disease prediction system suitable for various chronic diseases of healthy physical examination people in Shandong Province, and provide scientific basis for health intervention of the chronic diseases.

The other one is to analyze data of electronic health record and other data collected through examination through some methods, including human body measurement features (age, gender, body mass index and the like) and physiological record (including blood routine examination, blood glucose, routine urine examination and the like), and the dangerous factor of a certain disease is discovered by looking for the relation between the medical index and the chronic disease, so that the chronic disease is predicted. At the same time, some studies have explored the potential relation between the common dangerous factors and some common chronic diseases.

For example, Chinese patent document with the publication number CN107007284A discloses a multi-disease chronic disease information management system, including a database, an application server, several hospital clients and patient clients, wherein the database stores various physical examination data, doctor suggestion, health data reference range of various examination items and health state assessment index of patients; and the application server acquires various physical examination data and corresponding health data reference range, the health state assessment index of various chronic diseases and doctor suggestion of the specified patient in the database according to a first query instruction sent by the hospital/patient client to obtain the chronic disease assessment result, and returns the chronic disease assessment result of the current specified patient and the above various data to the hospital/patient client.

However, there is still no method to predict various chronic diseases at the same time by applying potential relations possibly existing among the various chronic diseases.

### SUMMARY OF THE INVENTION

The prevent invention provides a chronic disease prediction system based on a multi-task learning model, which is capable of predicting various chronic diseases at the same time by applying potential relations possibly existing among the various chronic diseases.

A chronic disease prediction system based on a multi-task learning model comprises a computer memory, a computer processor and a computer program which is stored in the computer memory and executable on the computer processor, wherein a trained chronic disease prediction model is stored in the computer memory, and the chronic disease prediction model is composed of a shared layer convolutional neural network and a plurality of chronic disease branch networks.

When executing the computer program, the computer processor implements the following steps:

preprocessing a to-be-predicted physical examination record and then inputting the record into the shared layer convolutional neural network of the chronic disease prediction model for feature extraction to obtain a feature map; and

inputting the obtained feature map into each chronic disease branch network and performing feature extraction and prediction respectively to obtain a chronic disease prediction result.

A structure of the shared layer convolutional neural network is as follows: firstly, through a multi-layer task shared convolutional layer, feature extraction is performed by using 3 and 6 convolutional cores with a size of 3*3, and a step length of the convolutional core is set as 1;

each chronic disease branch network is provided with 2 convolutional layers respectively, feature extraction is performed on each convolutional layer by 9 and 12 convolutional layers respectively, and step lengths of the convolutional layers are designed as 2 and 1 respectively; and finally, each branch sequentially passes through two full-connection layers with a node number of 32 and one softmax layer to obtain a final output.

The training process of the chronic disease prediction model is as follows:

acquiring chronic disease examination related physical examination data as sample data, labeling the sample data after preprocessing, and dividing the labeled sample data into a training set and a validation set by a five-fold cross validation method;

designing a data coding method for structured data in physical examination data to acquire input data of the chronic disease prediction data, wherein the data coding method comprises a content coding strategy and a spatial coding strategy, the content coding strategy being used to unify value types of data, and the spatial coding strategy being used to unify data formats the input model/data;

establishing a multi-task learning-based chronic disease prediction model, performing feature extraction and classification on the coded structured data by a deep learning method, and outputting prediction results of various chronic diseases at the same time; and

training the chronic prediction model by the training set, and adjusting parameters of the model according to the prediction result of the model and the coincidence degree of the label until the model converges.

Physical examination data used in the present invention is data in a csv format, and may also be structured data in other formats for a physical record of a patient. Each piece of csv data corresponding to a physical examination record of one patient, and each csv record comprises a plurality of physical examination index items. In the model training process, there may be some patients whose physical examination index items are missing, which will lead to large error and poor effect in model training. Therefore, in this step, these data records are eliminated. Meanwhile, some physical examination index items are missing in many patients, which will also lead to poor performance in the model training process. Therefore, these index items are eliminated.

Specifically, the preprocessing comprises: performing correlation analysis and missing value counting on various indexes in the physical examination data, eliminating data with missing values in a single record exceeding a certain ratio from the perspective of physical examination records, eliminating data indexes with missing values in all the records exceeding a certain ratio from the perspective of data indexes, grouping according to ages, and performing missing value filling on missing data in the physical examination records.

Specifically, patients are grouped according to their ages, and the missing item of data in each group is filled according to the average value or mode of the item in the group.

In order to improve the stability of the model performance, a five-fold cross validation method is selected and the data set is grouped, so that the training results of five different groups are averaged to reduce a variance, thereby reducing the sensitivity of the model performance on data division. The specific process of the five-fold cross validation method is as follows:

randomly dividing the sample data into five parts without repeated sampling, the number of each part of data samples being equal or close; and selecting one part as a test set at each time and the remaining four parts as the training set for model training, and repeating five times to make five different training set and validation set groups. Hence, each sub-set has a chance to serve as a validation set, and the rest of sets as training sets.

The content coding strategy adopts the following two specific operations:

coding text information in the physical examination record into numerical information by a label coding mode; and

coding a continuous variable in the physical examination record into a category variable by a one-hot coding mode to serve as input.

The specific operation process of the spatial coding strategy is as follows:

analyzing a correlation between any two of all variables in a one-dimensional vector, wherein the physical examination record after content coding is the one-dimensional vector; sorting in a descending order according to the sum of correlations between a certain variable and all other variables; and sequentially sorting all the variables after the descending sort to form a two-dimensional vector to serve as input data of a network.

The specific process of training the chronic disease prediction model by the training set is as follows:

inputting one group of training sets, and outputting a prediction result respectively through feature extraction of a shared layer with a potential correlation and feature extraction for a single chronic disease;

comparing the output prediction result with a label corresponding to data, applying an ACC (prediction accurate rate) function as loss of a current model and returning to the model, and updating parameters in the model;

when reaching a set ACC (prediction accurate rate) threshold or a specified number of iterations, stopping updating the model and outputting a result; and

sequentially inputting the remaining training sets by the above method for training until the model converges.

The training process further comprises: after each group of training sets are trained, inputting validation sets in the group into the model to obtain a corresponding classification result; and averaging loss values obtained by all the validation sets to serve as performance assessment of the model for finding an optimal parameter. Model performance assessment includes prediction accuracy on various single diseases.

Compared with the prior art, the present disclosure has the following beneficial effects:

the present invention builds the chronic disease prediction system based on the multi-task learning model. Firstly, data recorded by physical examination is preprocessed, and the data content and structure are coded, then a multi-task learning model is designed, feature extraction is performed on the potential relations possibly existing among various diseases by a multi-task shared layer, and feature extraction and final prediction are performed respectively through a single-task branch designed for single chronic disease, so that various chronic diseases can be predicted at the same time, and the potential relations possibly existing among various chronic diseases can be completely applied. In the training process, the model is trained by the five-fold cross validation method, and a stable effect and high accuracy rate can be achieved after many iterations.

### BRIEF DESCRIPTION OF THE DRAWINGS

FIG. 1 is a schematic diagram of a physical examination record preprocessing flow used by an embodiment of the present invention;

FIG. 2 is a schematic diagram of a five-fold cross validation method used in an embodiment of the present invention;

FIG. 3 is a flowchart of an overall framework of a network model according to the present invention;

FIG. 4 is an implementation method of a content coding strategy used in an embodiment of the present invention;

FIG. 5 is a schematic diagram of a network structure of a chronic disease prediction model used in an embodiment of the present invention; and

FIG. 6 is a result of model prediction in an embodiment of the present invention.

### DESCRIPTION OF THE EMBODIMENTS

The present invention is further described in detail below with reference to the accompanying drawings and embodiments. It should be noted that the following embodiments are intended to facilitate understanding of the present invention, without any limitation to the present invention.

A chronic disease prediction system based on a multi-task learning model comprises a computer memory, a computer processor and a computer program which is stored in the computer memory and executable on the computer processor, wherein a trained chronic disease prediction model is stored in the computer memory, and the chronic disease prediction model is composed of a shared layer convolutional neural network and a plurality of chronic disease branch networks. When executing the computer program, the computer processor implements the following steps:

a to-be-predicted physical examination record is preprocessed and then is input into the shared layer convolutional neural network of the chronic disease prediction model to perform feature extraction to obtain a feature map; and then the obtained feature map is input into each chronic disease branch network respectively to perform feature extraction and prediction respectively to obtain a chronic disease prediction result.

The following is the detailed instruction from the construction, training and validation processes of the model.

S01: a sample data set was established.

A physical examination data record was obtained and preprocessed, a sample data set was obtained from five cooperative hospitals, the sample data set totally comprises 48953 physical examination records, single physical examination record at most comprises 55 items of physical examination data, each physical examination item has different ranges of parameter references and also has some abnormal values, and each record was finely labeled by more than three professional doctors to distinguish whether the patient belongs to hypertension, diabetes, both hypertension and diabetes or was normal.

S02: a data set was preprocessed.

The obtained sample data set was preprocessed accordingly, and data was eliminated according to feature correlation and feature missing. Firstly, the correlation among all 55 indexes was analyzed. Considering the number of the indexes and the data coding mode in the present invention, in order to retain as much useful information as possible for each record and try not to increase redundant information, some variables were eliminated. According to the variable type corresponding to the value of each index, a correlation among the features was calculated by mainly using a Pearson correlation coefficient. For paired variables with a Pearson coefficient greater than 0.8, one feature with a large amount of missing data in the variable pair was eliminated. In addition, for all patients, if the feature missing amount was greater than 0.2, the data of the patient will be discarded. After elimination, there were totally 13358 physical examination records and 49 physical examination indexes in the data, and the missing amount of a value in each data variable was less than 0.2.

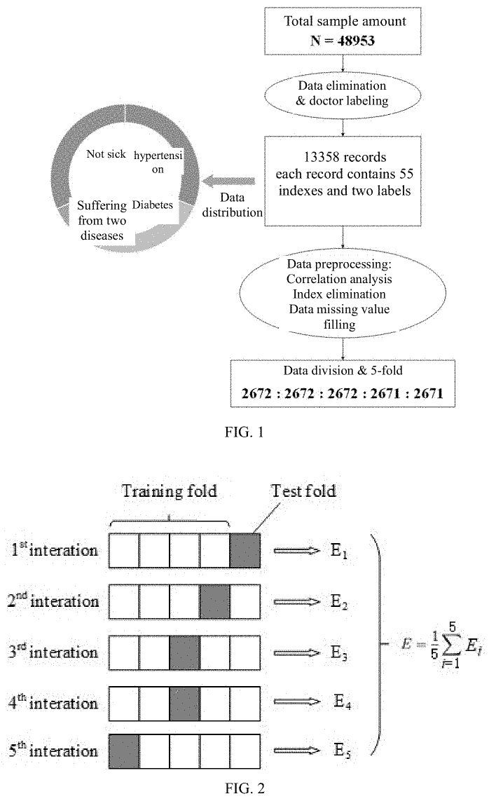

Then, these physical examination records were grouped according to ages for filling the missing data. Studies have shown that age was one of the dangerous factors for hypertension and diabetes. Therefore, age serves as an important grouping basis for filling the missing value. For different categories of data in the data set, firstly, the patients were divided into seven groups according to their ages. Then, for a certain feature to be filled, the model of the feature value in the group was selected for filling. The specific step of preprocessing the data set was as shown in FIG. 1.

The above sample data set was approximately and averagely divided into five parts for five-fold cross validation, wherein the number distribution of each part of data was [2672, 2672, 2672, 2671, 2671] and was respectively marked as [E1, E2, E3, E4, E5] for five times of model training and prediction, denoted as 1st iteration, 2nd iteration . . . . The process of the specific five-fold cross validation method was as shown in FIG. 2, wherein Training folds represents the training set, and Test folds represents the validation set.

S03: data was coded.

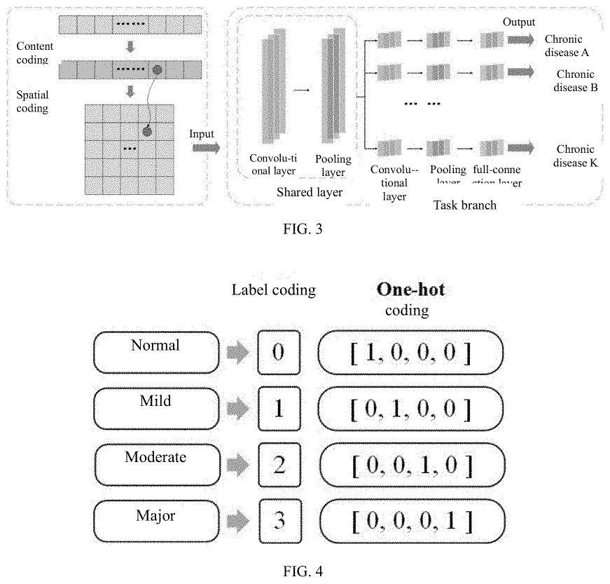

For 49 index items in each record, firstly, data of value bit text corresponding to the index item was coded, and the coding mode was as shown in FIG. 4. Then, the 49 index items were mapped to a 7*7 matrix by the spatial coding strategy as input of the network model, as shown in the left part in FIG. 3. The spatial mapping method here complies with the method described in the present invention. Firstly, a correlation between any two of the 49 index items was calculated respectively and was sorted in a descending order according to the sum of correlations between a certain index and all other indexes, so that a one-dimensional index sequence was mapped into a two-dimensional space, and the h-th value in the 49 indexes was mapped to the i, j-th position mij of a matrix M. (In one group of experiments, the same mapping mode was maintained, that is, certain indexes in one group of experiments in all samples were mapped to a fixed position, thereby ensuring the subsequent correlation analysis).

S04: a multi-task learning model (chronic disease prediction model) was built.

The chronic disease prediction model of the present invention takes a two-dimensional vector as an input, as shown in FIG. 3, firstly, a shared layer convolutional neural network shared by various diseases was designed, and feature extraction was performed on the potential correlations possibly existing among various diseases; and the feature maps after common feature extraction were subjected to feature extraction and prediction respectively through each branch for different chronic diseases.

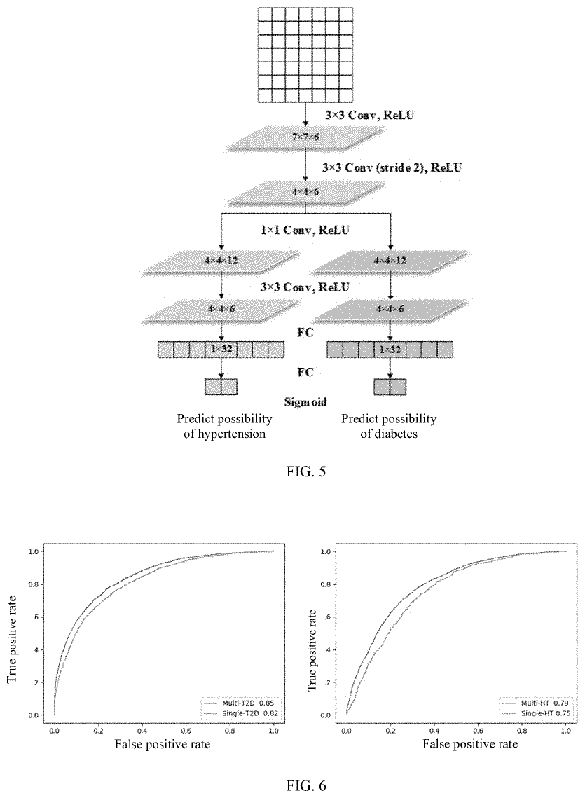

In this embodiment, a network model for two specific diseases such as diabetes and hypertension was built for performing feature extraction and disease prediction on the two diseases. The training data set in the I group of data after coding in the above step S03 was input into the model in individuals, that is, each input data was data of a two-dimensional matrix containing one physical examination record. Feature extraction and prediction were performed in the data input model, and the detailed structure of the model was as shown in FIG. 5. Firstly, through a two-layer task shared convolutional layer, feature extraction was performed by using 3 and 6 convolutional cores with a size of 3*3, and a step length of the convolutional core was set as 1. Then, feature extraction of diabetes physical examination data and feature extraction of hypertension physical examination data were performed respectively through a task specific branch in the model, two convolutional layers were designed for each branch, each convolutional layer was subjected to feature extraction respectively by 9 and 12 convolutional cores, and step lengths of the convolutional cores were designed as 2 and 1 respectively. Finally, two branches for predicting two diseases such as diabetes and hypertension sequentially pass through two full-connection layers with a node number of 32 and one softmax layer to obtain a final output. Each branch determines whether the patient suffers from diabetes and hypertension according to the feature extracted by the model respectively, wherein the branch 1 was relative to hypertension and the branch 2 was relative to diabetes. The determination result output by the model and a mark general cross entropy loss function corresponding to the physical examination marked by experts in the step 1 were subjected to loss calculation, and the sum of the loss values of the two branches serves as the loss function of the whole model for optimizing the model.

S05: test set data was predicted.

Data in the corresponding I group data test data set was input into the converged chronic disease prediction model based on multi-task learning trained in the step S04 to obtain a corresponding prediction result, all the test data in the group was subjected to ACC (prediction accurate rate) calculation, and the prediction accurate rate for hypertension and the prediction accurate rate for diabetes were calculated respectively.

S06: five-fold cross validation was performed.

The steps S04 and S05 were repeated for five times to complete five-fold cross validation to obtain the prediction accurate rates (respectively for hypertension and diabetes) on five test data sets, these prediction accurate rates were averaged to serve as performance assessment of the parameter and model, so that the optimal parameter was sought.

As shown in FIG. 6, after the model of the present invention was trained, the prediction accurate rate for hypertension can reach 73% and the prediction accurate rate for diabetes can reach 82%. Moreover, the AUC index can reach 79% and 85% or above, and compared with the single-mask model, the model has great advantages and better effect.

The above embodiments describe the technical solutions and beneficial effects of the present invention in detail. It should be understood that the above embodiments are only the specific embodiment of the present invention and are not used to limit the present invention. Any modification, supplement and equivalent substitution made within the principal scope of the present invention should be included in the protection scope of the present invention.

What is claimed is:

1. A chronic disease prediction system based on a multi-task learning model, comprising a computer memory, a computer processor and a computer program which is stored in the computer memory and executable on the computer processor, wherein a trained chronic disease prediction model is stored in the computer memory, and the chronic disease prediction model is composed of a shared layer convolutional neural network and a plurality of chronic disease branch networks; and
when executing the computer program, the computer processor implements the following steps:
preprocessing a to-be-predicted physical examination record and then inputting the record into the shared layer convolutional neural network of the chronic disease prediction model for feature extraction to obtain a feature map, and
inputting the obtained feature map into each chronic disease branch network and performing feature extraction and prediction respectively to obtain a chronic disease prediction result.

2. The chronic disease prediction system based on the multi-task learning model according to claim 1, wherein a structure of the shared layer convolutional neural network is as follows: firstly, through a multi-layer task shared convolutional layer, feature extraction is performed by using 3 and 6 convolutional cores with a size of 3*3, and a step length of the convolutional core is set as 1;
each chronic disease branch network is provided with 2 convolutional layers respectively, feature extraction is performed on each convolutional layer by 9 and 12 convolutional layers respectively, and step lengths of the convolutional layers are designed as 2 and 1 respectively; and finally, each branch sequentially passes through two full-connection layers with a node number of 32 and one softmax layer to obtain a final output.

3. The chronic disease prediction system based on the multi-task learning model according to claim 1, wherein the training process of the chronic disease prediction model is as follows:
acquiring chronic disease examination related physical examination data as sample data, labeling the sample data after preprocessing, and dividing the labeled sample data into a training set and a validation set by a five-fold cross validation method;
designing a data coding method for structured data in physical examination data to acquire input data of the chronic disease prediction data, the data coding method comprising a content coding strategy and a spatial coding strategy, the content coding strategy being used to unify value types of data, and the spatial coding strategy being used to unify data formats the input type;
establishing a multi-task learning-based chronic disease prediction model, performing feature extraction and classification on the coded structured data by a deep learning method, and outputting prediction results of various chronic diseases at the same time; and
training the chronic prediction model by the training set, and adjusting parameters of the model according to the prediction result of the model and the coincidence degree of the label until the model converges.

4. The chronic disease prediction system based on the multi-task learning model according to claim 3, wherein the preprocessing comprises: performing correlation analysis and missing value counting on various indexes in the physical examination data, eliminating data with missing values in a single record exceeding a certain ratio from the perspective of physical examination records, eliminating data indexes with missing values in all the records exceeding a certain ratio from the perspective of data indexes, grouping according to ages, and performing missing value filling on missing data in the physical examination records.

5. The chronic disease prediction system based on the multi-task learning model according to claim 3, wherein the specific process of the five-fold cross validation method is as follows:
randomly dividing the sample data into five parts without repeated sampling, the number of each part of data samples being equal or close; and selecting one part as a test set at each time and the remaining four parts as the training set for model training, and repeating five times to make five different training set and validation set groups.

6. The chronic disease prediction system based on the multi-task learning model according to claim 3, wherein the content coding strategy adopts the following two specific operations:
coding text information in the physical examination record into numerical information by a label coding mode; and
coding text information in the physical examination record into numerical information by a one-hot coding mode to serve as input.

7. The chronic disease prediction system based on the multi-task learning model according to claim 3, wherein the specific process of the spatial coding strategy is as follows:
analyzing a correlation between any two of all variables in a one-dimensional vector, wherein the physical examination record after content coding is the one-dimensional vector; sorting in a descending order according to the sum of correlations between a certain variable and all other variables; and sequentially sorting all the variables after the descending sort to form a two-dimensional vector to serve as input data of a network.

8. The chronic disease prediction system based on the multi-task learning model according to claim 3, wherein the specific process of training the chronic disease prediction model by the training set is as follows:
inputting one group of training sets, and outputting a prediction result respectively through feature extraction of a shared layer with a potential correlation and feature extraction for a single chronic disease;
comparing the output prediction result with a label corresponding to data, applying an ACC function as loss of a current model and returning to the model, and updating parameters in the model;
when reaching a set ACC threshold or a specified number of iterations, stopping updating the model and outputting a result; and
sequentially inputting the remaining training sets by the above method for training until the model converges.

9. The chronic disease prediction system based on the multi-task learning model according to claim 8, wherein the training process further comprises: after each group of training sets are trained, inputting validation sets in the group into the model to obtain a corresponding classification result; and averaging loss values obtained by all the validation sets to serve as performance assessment of the model for finding an optimal parameter.
